(19) United States
(12) Patent Application Publication
(10) Pub. No.: US 2018/0000811 A1
(43) Pub. Date: Jan. 4, 2018

(54) STABLE PHARMACEUTICAL COMPOSITIONS COMPRISING ANTIBACTERIAL AGENT

(71) Applicant: WOCKHARDT LIMITED, Chikalthana, Aurangabad (IN)

(72) Inventors: Ajmal Shareef MOHAMMAD, Bihar, 1, Bhagalpur (IN); Vikranth Kumar BASETTY, Telangana state Karimnagar (IN); Mrutunjaya SAHU, Orissa Pin 3 Ganjam (IN); Manohar Vishwanath LALGE, Mumbai (IN)

(73) Assignee: WOCKHARDT LIMITED, Chikalthana, Aurangabad (IN)

(21) Appl. No.: 15/546,371

(22) PCT Filed: May 7, 2016
(86) PCT No.: PCT/IB2016/052614

§ 371 Date: Jul. 26, 2017

(30) Foreign Application Priority Data
May 8, 2015 (IN) 1839/MUM/2015

(51) Int. Cl. A61K 31/473; A61K 9/00; A61K 47/18; C07D 455/06; A61K 9/20

(52) U.S. Cl. CPC A61K 31/473; C07D 455/06; A61K 9/2059; A61K 47/183; A61K 9/0002; A61K 9/2054; A61K 9/2027; A61K 9/2013; A61K 2300/00

(57) ABSTRACT

Stable pharmaceutical compositions comprising a compound of Formula (I) or a stereoisomer or a pharmaceutically acceptable derivative thereof are disclosed. Formula (I)

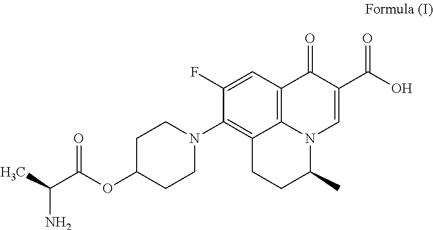 [SMILES: C[C@H](N)C(=O)OC1CCN(c2c(F)cc3c(=O)c(C(=O)O)cn4c3c2CC[C@@H]4C)CC1]

### PRIORITY APPLICATION(S)

This application claims priority to Indian Patent Application No. 1839/MUM/2015 filed on May 8, 2015, the disclosures of which is incorporated herein by reference in its entirety as if fully rewritten herein.

### FIELD OF THE INVENTION

The invention relates to stable pharmaceutical compositions of L-alanine, 1-[(5S)-2-carboxy-9-fluoro-6,7-dihydro-5-methyl-1-oxo-1H,5H-benzo[i,j]quinolizin-8-yl]-4-piperidinyl ester or a stereoisomer, or a pharmaceutically acceptable derivative thereof. The invention also relates to the preparation of such compositions and their use in preventing or treating infections.

### BACKGROUND OF THE INVENTION

Bacterial infections continue to remain one of the major causes contributing towards human diseases. Various benzoquinolizine-2-carboxylic acid compounds have been disclosed to possess potent antibacterial activity. A compound of Formula (I), chemically known as L-alanine, 1-[(5S)-2-carboxy-9-fluoro-6,7-dihydro-5-methyl-1-oxo-1H,5H-benzo[i,j]quinolizin-8-yl]-4-piperidinyl ester has antibacterial properties and is disclosed in U.S. Pat. No. 6,750,224. U.S. Pat. No. 7,868,173 discloses various sulfonic acid salts of a compound of Formula (I). WO2008053298 discloses the pharmaceutical compositions of benzoquinolizine-2-carboxylic acid. The present invention describes the stable oral pharmaceutical compositions of a compound of Formula (I) or a stereoisomer or a pharmaceutically acceptable derivative thereof.

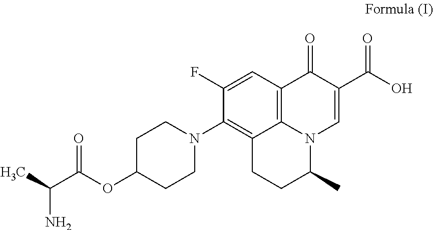 [SMILES: C[C@H](N)C(=O)OC1CCN(c2c(F)cc3c(=O)c(C(=O)O)cn4c3c2CC[C@@H]4C)CC1]

### SUMMARY OF THE INVENTION

Accordingly, there are provided stable pharmaceutical compositions comprising a compound of Formula (I) or a stereoisomer or a pharmaceutically acceptable derivative thereof, their methods for preparation, and their use in treating or preventing bacterial infections.

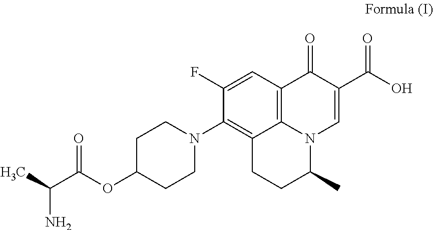 [SMILES: C[C@H](N)C(=O)OC1CCN(c2c(F)cc3c(=O)c(C(=O)O)cn4c3c2CC[C@@H]4C)CC1]

In one general aspect, there are provided stable pharmaceutical compositions comprising a compound of Formula (I) or a stereoisomer or a pharmaceutically acceptable derivative thereof, and one or more pharmaceutically acceptable excipients.

In another general aspect, there are provided stable pharmaceutical compositions comprising L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate or a stereoisomer thereof; and one or more pharmaceutically acceptable excipients.

In another general aspect, there are provided stable pharmaceutical compositions comprising a compound of Formula (I) or a stereoisomer or a pharmaceutically acceptable derivative thereof, wherein said compositions are adapted for oral administration.

In another general aspect, there are provided stable pharmaceutical compositions comprising a compound of Formula (I) or a stereoisomer or a pharmaceutically acceptable derivative thereof, wherein said compositions are formulated as tablets.

In another general aspect, there are provided pharmaceutical compositions for use in treatment or prevention of bacterial infections.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The present invention provides stable pharmaceutical compositions comprising a compound of Formula (I) or a stereoisomer or a pharmaceutically acceptable derivative thereof, their methods for preparation, and their use in treating or preventing bacterial infections.

The term “stereoisomers” as used herein refers to compounds that have identical chemical constitution, but differ with regard to the arrangement of their atoms or groups in space. The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, within the scope of the invention. In general, a reference to a compound is intended to cover its stereoisomers and mixture of various stereoisomers.

The term “pharmaceutically acceptable derivative” as used herein refers to and includes any pharmaceutically acceptable salt, pro-drugs, metabolites, esters, ethers, hydrates, polymorphs, solvates, complexes and adducts of a compound described herein which, upon administration to a subject, is capable of providing (directly or indirectly) the parent compound. For example, the term “antibacterial agent or a pharmaceutically acceptable derivative thereof” includes all derivatives of the antibacterial agent (such as salts, pro-drugs, metabolites, esters, ethers, hydrates, polymorphs, solvates, complexes and adducts) which, upon administration to a subject, are capable of providing (directly or indirectly) the antibacterial agent.

The term “pharmaceutically acceptable salt” as used herein refers to one or more salts of a given compound which possesses the desired pharmacological activity of the free compound and which are neither biologically nor otherwise undesirable. In general, the term “pharmaceutically acceptable salts” refer to salts that are suitable for use in contact with the tissues of human and animals without undue toxicity, irritation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutically acceptable salts in details. Compound of Formula (I) can be used as such or in the form of its suitable salt. Typical, non-limiting examples of such salts include sulfonic acid salts. A reference to compound of Formula (I) is intended to include reference to such salts as well.

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of normal floras, which are not desirable. The term “infection” includes infection caused by bacteria.

The term “subject” as used herein refers to vertebrate or invertebrate, including a mammal. The term “subject” includes human, animal, a bird, a fish, or an amphibian. Typical, non-limiting examples of a “subject” includes humans, cats, dogs, horses, sheep, bovine cows, pigs, lambs, rats, mice and guinea pigs.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions or one or more of pharmaceutically active ingredients discussed herein, with or without additional pharmaceutically active or inert ingredients, in order to: (i) reduce or eliminate either a bacterial infection or one or more symptoms of the bacterial infection, or (ii) retard the progression of a bacterial infection or one or more symptoms of the bacterial infection, or (iii) reduce the severity of a bacterial infection or of one or more symptoms of the bacterial infection, or (iv) suppress the clinical manifestation of a bacterial infection, or (v) suppress the manifestation of adverse symptoms of the bacterial infection.

The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the type/nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention includes oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop or mouthwash. In case of a pharmaceutical composition comprising more than one ingredients (active or inert), one of the way of administering such composition is by admixing the ingredients (e.g. in the form of a suitable unit dosage form such as tablet, capsule, solution, powder or like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “pharmaceutically inert ingredient” or “carrier” or “excipient” refers to a compound or material used to facilitate administration of a compound, for example, to increase the solubility of the compound. Typical, non-limiting examples of solid carriers include, starch, lactose, dicalcium phosphate, sucrose, and kaolin. Typical, non-limiting examples of liquid carriers include sterile water, saline, buffers, non-ionic surfactants, and edible oils such as peanut oil and sesame oils. In addition, various adjuvants commonly used in the art may also be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press., which is incorporated herein by reference in its entirety.

The term “about” as used in herein means within a acceptable error range for the particular value as determined by one of the ordinary skilled in the art, which will depend in part on how the value is measured or determined. Alternatively, “about” with respect to the compositions can mean plus or minus a range of up to 10%.

The term “related substances” as used herein refers to one or more impurities present in the pharmaceutical composition according to the invention. Such impurities may be present in the composition due to degradation of one or more components in the composition, for example the active or inactive ingredients.

The amount of impurities is calculated on the basis of the compound of Formula (I) or a stereoisomer or a pharmaceutically acceptable derivative thereof present in the composition.

The term “Substance A” as used herein refers to a compound which is “S-(−)-9-fluoro-8-(4-hydroxy-piperidin-1-yl)-5-methyl-6,7-dihydro-1-oxo-1H,5H-benzo[i,j] quinolizine-2-carboxylic acid”.

The term “Substance B” as used herein refers to a compound which is “(S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt”.

The term “Substance C” as used herein refers to a compound which is “(S)-(−)-8-(4-D-alaninyl oxypiperidin-1-yl)-5-methyl-6,7-dihydro-1-oxo-1H,5H-benzo[i,j] quinolizine-2-carboxylic acid, methane sulfonic acid salt”.

The term “Substance D” as used herein refers to a compound which is “(S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j] quinolizine-2-carboxylic acid”.

The term “stable pharmaceutical composition” as used herein refers to a composition which is stable over extended period of time on storage as assessed from the content of one or more impurities in the composition. The term “stable pharmaceutical composition” as used herein includes composition comprising a compound of Formula (I) or a stereoisomer or a pharmaceutically acceptable derivative thereof; said composition comprising one or more of the following:
(a) less than about 2% w/w of total impurity following storage for six months at a temperature of 40° C. and a relative humidity of 75%;
(b) less than about 2% w/w of S-(−)-9-fluoro-8-(4-hydroxy-piperidin-1-yl)-5-methyl-6,7-dihydro-1-oxo-1H,5H-benzo[i,j] quinolizine-2-carboxylic acid following storage for six months at a temperature of 40° C. and a relative humidity of 75%;
(c) less than about 0.5% w/w of (S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%;
(d) less than about 0.5% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%; or
(e) less than about 0.1% w/w of (S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j] quinolizine-2-carboxylic acid following storage for six months at a temperature of 40° C. and a relative humidity of 75%.

In one general aspect, there are provided stable pharmaceutical compositions comprising a compound of Formula (I) or a stereoisomer or a pharmaceutically acceptable derivative thereof, and one or more pharmaceutically acceptable excipients.

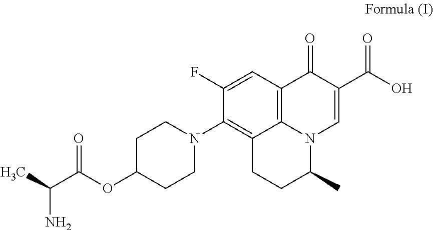 [SMILES: C[C@H](N)C(=O)OC1CCN(c2c(F)cc3c(=O)c(C(=O)O)cn4c3c2CC[C@@H]4C)CC1]

In some embodiments, compound of Formula (I) is present as L-alanine, 1-[(5S)-2-carboxy-9-fluoro-6,7-dihydro-5-methyl-1-oxo-1H,5H-benzo[i,j]quinolizin-8-yl]-4-piperidinyl ester or a stereoisomer or a pharmaceutically acceptable derivative thereof.

In some embodiments, compound of Formula (I) is present as L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate (also known as (2′S,5S)-9-fluoro-6,7-dihydro-8-(4-L-alaninyl-oxy-piperidin-1-yl)-5-methyl-1-oxo-1H,5H-benzo[i,j]quinolizine-2-carboxylic acid methanesulfonic acid salt).

In some embodiments, compound of Formula (I) is present as:

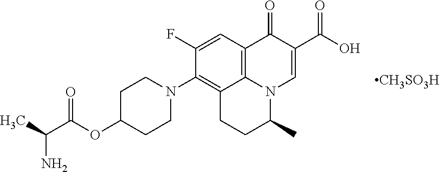

In some embodiments, there are provided stable pharmaceutical compositions comprising L-alanine, 1-[(5S)-2-carboxy-9-fluoro-6,7-dihydro-5-methyl-1-oxo-1H,5H-benzo[i.j]quinolizin-8-yl]-4-piperidinyl ester or a stereoisomer or a pharmaceutically acceptable derivative thereof.

In some other embodiments, there are provided stable pharmaceutical compositions comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate.

In some embodiments, there are provided stable pharmaceutical compositions comprising a compound of Formula (I) or a stereoisomer or a pharmaceutically acceptable derivative thereof, wherein the compound of Formula (I) or a stereoisomer or a pharmaceutically acceptable derivative thereof is present in the composition in an amount of about 0.1 gram to about 10 gram.

In some embodiments, compositions according to the invention are adapted for oral administration.

In some other embodiments, compositions according to invention are present as immediate release dosage form.

In some embodiments, the compositions according to the invention comprise less than about 2% w/w of total impurities following storage for six months at a temperature of 40° C. and a relative humidity of 75%.

In some embodiments, the compositions according to invention comprise less than about 2% w/w of S-(−)-9-fluoro-8-(4-hydroxy-piperidin-1-yl)-5-methyl-6,7-dihydro-1-oxo-1H,5H-benzo[i,j] quinolizine-2-carboxylic acid following storage for six months at a temperature of 40° C. and a relative humidity of 75%.

In some embodiments, the compositions according to invention comprise less than about 0.5% w/w of (S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%.

In some embodiments, the compositions according to invention comprise less than about 0.5% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%. In some other embodiments, the compositions according to invention comprise less than about 1% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%.

In some embodiments, the compositions according to invention comprise less than about 0.1% w/w of (S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j]quinolizine-2-carboxylic acid following storage for six months at a temperature of 40° C. and a relative humidity of 75%.

In some embodiments, the pharmaceutical compositions according to the invention comprise the following:

(i) less than about 2% w/w of S-(−)-9-fluoro-8-(4-hydroxy-piperidin-1-yl)-5-methyl-6,7-dihydro-1-oxo-1H,5H-benzo[i,j] quinolizine-2-carboxylic acid;

(ii) less than about 0.5% w/w of (S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt;

(iii) less than about 0.5% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt; and

(iv) less than about 0.1% w/w of (S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j]quinolizine-2-carboxylic acid;

following storage for six months at a temperature of 40° C. and a relative humidity of 75%.

In some embodiments, there is provided stable pharmaceutical composition comprising compound of Formula (I) or a stereoisomer or a pharmaceutically acceptable derivative thereof, wherein the composition exhibits a dissolution profile such that about 50% or more of a compound of Formula (I) or a stereoisomer or a pharmaceutically acceptable derivative thereof is released within 15 minutes, when measured using a USP Dissolution Apparatus II in 900 ml of 0.1 N HCl at a temperature of 37±0.5° C. and 50 rpm.

In some other embodiments, there is provided stable pharmaceutical composition comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate, wherein the composition exhibits a dissolution profile such that about 75% or more of a compound of Formula (I) or a stereoisomer or a pharmaceutically acceptable derivative thereof is released within 20 minutes, when measured using a USP Dissolution Apparatus II in 900 ml of 0.1 N HCl at a temperature of 37±0.5° C. and 50 rpm.

In some other embodiments, there is provided stable pharmaceutical composition comprising compound of Formula (I) or a stereoisomer or a pharmaceutically acceptable derivative thereof, wherein the composition exhibits a dissolution profile such that about 75% or more of a compound of Formula (I) or a stereoisomer or a pharmaceutically acceptable derivative thereof is released within 20 minutes, when measured using a USP Dissolution Apparatus II in 900 ml of 0.1 N HCl at a temperature of 37±0.5° C. and 50 rpm.

The pharmaceutical compositions according to the invention may include one or more pharmaceutically acceptable carriers or excipients or the like. Typical, non-limiting examples of such carriers or excipients include diluents, disintegrants, binders, wetting agents, emulsifying agents, solubilizing agents, buffering agents, glidants, lubricants, preservatives, stabilizing agents, flavoring agents and the like.

In some embodiments, there are provided pharmaceutical compositions comprising a compound of Formula (I) or a stereoisomer or a pharmaceutically acceptable derivative thereof as an active ingredient and one or more excipients selected from diluent, disintegrant, binder, lubricant or glidant.

The pharmaceutical compositions to the invention may be formulated into a variety of solid oral dosage forms. Typical, non-limiting examples of some oral dosage forms include tablet, capsule, powder, discs, caplets, pellets, granules, granules in capsule, minitablets, minitablets in capsule, pellets in capsule and the like. In some embodiments, the compositions according to invention may also be formulated into other dosage form suitable for oral administration such as suspensions, emulsions, syrups, elixirs and the like.

In some embodiments, compound of Formula (I) or a stereoisomer or a pharmaceutically acceptable derivative thereof present in the composition is in an amount within the range of from about 10% to about 90% by weight.

In some embodiments, L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate present in the composition is in an amount within the range of from about 10% to about 90% by weight.

In some embodiments, diluent is present in an amount within the range of from about 1% to about 80% by weight. In some other embodiments, diluent is present in an amount within the range of from about 1% to about 30% by weight.

In some embodiments, disintegrant, if present, is present in an amount within the range of from about 0% to about 30% by weight. In some other embodiments, disintegrant is present in an amount within the range of from about 1% to about 15% by weight.

In some embodiments, binder, if present, is present in an amount within the range of from about 0% to about 30% by weight. In some other embodiments, binder is present in an amount within the range of from about 0.25% to about 10% by weight.

In some embodiments, glidant, if present, is present in an amount within the range of from about 0% to about 20% by weight. In some other embodiments, glidant is present in an amount within the range of from about 0.25% to about 10% by weight.

In some embodiments, lubricant, if present, is present in an amount within the range of from about 0% to about 20% by weight. In some other embodiments, lubricant is present in an amount within the range of from about 0.25% to about 5% by weight.

In some embodiments, the formulated tablets are coated with a suitable coating material dissolved in a suitable solvent. In some embodiments, coating is present in an amount within the range of from about 0.25% to about 5% by weight.

In some embodiments, there is provided a pharmaceutical composition comprising:

a compound of Formula (I) or a stereoisomer or a pharmaceutically acceptable derivative thereof as an active ingredient in an amount between about 10% to about 90% by weight;

at least one or more diluent in an amount between about 1% to about 30% by weight;

optionally one or more disintegrant in an amount between about 1% to about 15% by weight;

optionally one or more binder selected in an amount between about 0.25% to about 10% by weight;

optionally one or more lubricant in an amount between about 0.25% to about 5% by weight;

optionally one or more glidant in an amount between about 0.25% to about 10% by weight;

optionally film coating in an amount between about 0.25% to about 5% by weight.

In some embodiments, there is provided a pharmaceutical composition comprising:

L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate in an amount between about 10% to about 90% by weight;

at least one or more diluent in an amount between about 1% to about 30% by weight;

optionally one or more disintegrant in an amount between about 1% to about 15% by weight;

optionally one or more binder selected in an amount between about 0.25% to about 10% by weight;

optionally one or more lubricant in an amount between about 0.25% to about 5% by weight;

optionally one or more glidant in an amount between about 0.25% to about 10% by weight;

optionally film coating in an amount between about 0.25% to about 5% by weight.

Typical, non-limiting examples of diluents include microcrystalline cellulose, cellulose, lactose, starch, pregelatinized starch, corn starch, calcium carbonate, calcium sulfate, sugar, dextrates, sucrose, dextrin, fructose, dextrose, xylitol, polysaccharide, dibasic calcium phosphate dihydrate, tribasic calcium phosphate, calcium sulphate, kaolin, magnesium carbonate, magnesium oxide, maltodextrin, mannitol, polymethacrylates, potassium chloride, sodium chloride, sorbitol, and the like.

Typical, non-limiting example of binders include acacia, alginic acid, carbomer (carbopol), carboxymethylcellulose sodium, corn starch, dextrin, ethyl cellulose, methyl cellulose, gelatin, guar gum, hydrogenated vegetable oil, hydroxyethyl cellulose, hydroxypropylcellulose, hydroxypropylmethylcellulose, liquid glucose, magnesium aluminium silicate, maltodextrin, methyl cellulose, cellulose acetate, polymethacrylates, povidone, polyvinyl alcohol, pregelatinized starch, sodium alginate, starch, carnuba wax, paraffin, spermaceti, polyethylenes, microcrystalline wax and the like.

Typical, non-limiting examples of disintegrants include alginic acid, carboxymethylcellulose calcium, carboxymethylcellulose sodium, colloidal silicon dioxide, croscarmellose sodium, crospovidone, gura gum, low substituted hydroxypropyl cellulose, magnesium aluminium silicate, methyl cellulose, microcrystalline cellulose, polacrilin potassium, powdered cellulose, starch, pregelatinized starch, corn starch, potato starch, sodium alginate, sodium starch glycolate, and the like.

Typical, non-limiting examples of glidants include silicon dioxide, colloidal silicon dioxide, magnesium silicate, magnesium trisilicate, powdered cellulose, starch, talc, tribasic calcium phosphate and the like.

Typical non-limiting examples of lubricants include magnesium stearate, zinc stearate, calcium stearate, carnauba wax, palmitic acid, glyceryl monosterate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, myristic acid, talc, zinc stearate and the like.

In some embodiments, the compositions according to invention are coated with suitable coating polymers. Typical non-limiting examples of coating polymers include hydroxypropylmethyl cellulose, polyvinyl alcohol, ethyl cellulose, methacrylic polymers, hydroxypropyl cellulose, starch and the like. In some embodiments, coating can optionally include a plasticizer. Typical, non-limiting examples of plasticizers include triacetin, diethyl phthalate, tributyl sebacate, polyethylene glycol, glycerin, triacetin, triethyl citrate and the like. In some embodiments, coating can also optionally include an anti-adherent or glidant. Typical, non-limiting examples of anti-adherent or glidant include talc, fumed silica, magnesium stearate and the like. In some other embodiments, coating can also optionally include an opacifier. Typical, non-limiting example of opacifier includes titanium dioxide and the like. In yet another embodiment, coating can also optionally include one or more colorants. In some embodiments, the compositions according to present invention are film coated with a suitable opadry coating material.

In some embodiments, there is provided a pharmaceutical composition comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate and optionally one or more pharmaceutically acceptable excipients selected from diluent, binder, disintegerant, lubricant or glidant; wherein amount of disintegerant is less than 10% by weight of the composition.

In some embodiments, there is provided a pharmaceutical composition comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate and optionally one or more pharmaceutically acceptable excipients selected from diluent, binder, disintegerant, lubricant or glidant; wherein the composition is free of lactose.

In some embodiments, there is provided a pharmaceutical composition comprising:

L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate in an amount between about 10% to about 90% by weight;

Microcrystalline cellulose in an amount of between about 1% to about 10% by weight;

Crosscamellose sodium in an amount between about 1% to about 10% by weight;

Povidone in an amount between about 0.25% to about 5% by weight;

Talc in an amount between about 0.25% to about 5% by weight;

Sodium stearyl fumarate in an amount between about 0.25% to about 5% by weight;

Opadry coating in an amount between about 0.25% to about 5% by weight.

In some embodiments, there is provided a pharmaceutical composition comprising: about 200 to about 1000 mg of L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate; about 10 mg to about 100 mg of microcrystalline cellulose; about 10 mg to about 100 mg of crosscarmellose sodium; about 1 mg to about 25 mg of povidone, about 1 mg to about 25 mg of talc; about 1 mg to about 15 mg of sodium stearyl fumarate and about 1 mg to about 50 mg of opadry coating.

In some embodiments, there is provided a pharmaceutical composition comprising: about 293.095 mg of L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate; about 21.55 mg of microcrystalline cellulose; about 23.5 mg of crosscarmellose sodium; about 7.0 mg of povidone, about 6.5 mg of talc; about 3.35 mg of sodium stearyl fumarate and about 10.65 mg of opadry coating.

In some embodiments, there is provided a pharmaceutical composition comprising: about 586.19 mg of L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate; about 53.11 mg of microcrystalline cellulose; about 47.0 mg of crosscarmellose sodium; about 14.0 mg of povidone, about 13.0 mg of talc; about 6.7 mg of sodium stearyl fumarate and about 21.60 mg of opadry coating.

In some embodiments, there is provided a pharmaceutical composition comprising: about 732.738 mg of L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate; about 66.387 mg of microcrystalline cellulose; about 58.75 mg of crosscarmellose sodium; about 17.5 mg of povidone, about 16.25 mg of talc; about 8.375 mg of sodium stearyl fumarate and about 27 mg of opadry coating.

In some embodiments, the active ingredient in the compositions according to the invention has 90% of the particles smaller than 150 micrometers (d₉₀ is 150 μm). In some other embodiment, the active ingredient in the compositions according to the invention has 50% of the particles smaller than 60 micrometers (d₅₀ is 60 μm).

In some embodiments, there is provided a stabilized solid pharmaceutical composition comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate having d₉₀ particle size equal to or less than 150 μm.

In some other embodiments, there is provided stable pharmaceutical composition comprising L-alanine, 1-[(5S)-2-carboxy-9-fluoro-6,7-dihydro-5-methyl-1-oxo-1H,5H-benzo[i,j] quinolizin-8-yl]-4-piperidinyl ester, methanesulfonate having d₉₀ particle size equal to or less than 150 μm, wherein the composition exhibits a dissolution profile such that about 75% or more of a compound of Formula (I) or a stereoisomer or a pharmaceutically acceptable derivative thereof is released within 20 minutes, when measured using a USP Dissolution Apparatus II in 900 ml of 0.1 N HCl at a temperature of 37±0.5° C. and 50 rpm.

In some embodiments, the compositions according to the invention are formulated as tablets. Such tablets may be prepared using known techniques. In some embodiments, the compositions according to the invention are formulated as tablets by following dry granulation, wet granulation or direct compression techniques. In some embodiments, compositions according to the invention are formulated as tablets by following a wet granulation technique.

In some embodiments, there is provided a process for preparing the composition according to invention in the form of tablets; said process comprising:
(a) mixing a compound of Formula (I) or a stereoisomer or a pharmaceutically acceptable derivative thereof with one or more diluents and one or more disintegrants;
(b) wet granulating the mixture of step (a) in presence of a binder solution;
(c) drying and sieving the granulated mixture obtained in step (b);
(d) optionally blending the granulated mixture obtained in step (c) with one or more of a diluent, binder, disintegrant, glidant and lubricant;
(e) compressing the mixture obtained in step (c) or step (d) into tablets; and
(f) optionally film coating the tablets.

In some other embodiments, there is provided a process for preparing the composition according to invention in the form of tablets; said process comprising:
(a) mixing L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate with one or more diluents and one or more disintegrants;
(b) wet granulating the mixture of step (a) in presence of a binder solution;
(c) drying and sieving the granulated mixture obtained in step (b);
(d) optionally blending the granulated mixture obtained in step (c) with one or more of a diluent, binder, disintegrant, glidant and lubricant;
(e) compressing the mixture obtained in step (c) or step (d) into tablets; and
(f) optionally film coating the tablets.

In some embodiments, the compositions according to invention are formulated as granules or granules in capsules. In some embodiments, there is provided a process for preparing the composition according to invention in the form of granules; said process comprising:
(a) mixing a compound of Formula (I) or a stereoisomer or a pharmaceutically acceptable derivative thereof with one or more diluents and one or more disintegrants;
(b) wet granulating the mixture of step (a) in presence of a binder solution;
(c) drying and sieving the granulated mixture obtained in step (b); and
(d) optionally blending the granulated mixture obtained in step (c) with one or more of a diluent, binder, disintegrant, glidant and lubricant.

In some embodiments, the pharmaceutical compositions according to the invention are used in treatment or prevention of bacterial infections.

In some other embodiments, there are provided methods for treating or preventing bacterial infections in a subject, wherein said method comprises administering to said subject a pharmaceutical composition comprising a compound of Formula (I) or a stereoisomer or a pharmaceutically acceptable derivative thereof.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### Examples

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

The pharmaceutical compositions according to invention are formulated as tablets. Table 1 provides the qualitative and quantitative compositions according to the invention.

Manufacturing Procedure:

The compound of Formula (I) in form of its mesylate salt (L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate), microcrystalline cellulose, crosscarmellose sodium were weighed, sifted, and mixed in Rapid Mixer Granulator. The above mass was granulated by spraying aqueous solution of povidone. The granules were dried in a fluidized bed drier, sifted and milled. The resulting granules were blended with sifted microcrystalline cellulose, crosscarmellose sodium, talc and sodium stearyl fumarate. The lubricated granules were compressed into tablets using suitable tooling. The tablets were coated with aqueous dispersion of opadry.

TABLE 1
Pharmaceutical compositions according to the invention

| | | mg/Tablet | | |
|---|---|---|---|---|
| | | Example | Example | Example |
| Sr. | Ingredients | 1 | 2 | 3 |

INTRAGRANULAR

| | | | | |
|---|---|---|---|---|
| 1 | Compound of Formula (I) | 293.095 | 586.190 | 732.738 |
| | (as a mesylate salt) | | | |
| 2 | Microcrystalline Cellulose | 16.55 | 50.010 | 62.512 |
| | (Avicel PH 101) | | | |
| 3 | Croscarmesllose Sodium | 10.00 | 20.000 | 25.000 |
| | (Ac-Di-Sol) | | | |
| 4 | Povidone K30 | 7.0 | 14.000 | 17.500 |
| | (Kollidone K30) | | | |
| 5 | Purified water USP | q.s | q.s. | q.s. |

EXTRAGRANULAR

| | | | | |
|---|---|---|---|---|
| 6 | Microcrystalline cellulose | 5.00 | 3.100 | 3.875 |
| | (Avicel PH 102) | | | |
| 7 | Croscarmellose Sodium | 13.5 | 27.000 | 33.750 |
| | (Ac-Di-Sol) | | | |
| 8 | Talc | 6.5 | 13.000 | 16.250 |
| 9 | Sodium steryl fumarate | 3.350 | 6.700 | 8.375 |
| | (Pruv) | | | |

| | | | |
|---|---|---|---|
| CORE TABLET (mg) | 355.000 | 720.000 | 900.000 |

FILM COATING

| | | | | |
|---|---|---|---|---|
| 10 | Opadry Yellow | 10.650 | 21.600 | 27.000 |
| | (03F52057) | | | |
| 11 | Purified Water USP | q.s | q.s. | q.s. |
| | Total (Coated Tablet Weight) | 365.65 | 741.600 | 927.000 |
| | mg | | | |

The compositions according to invention were tested for their in-vitro release profile of an active ingredient. Table 2 provides the dissolution profile for the tablets comprising a compound of Formula (I) (as mesylate salt) prepared as per compositions given in Table 1. The drug release rate was determined using USP Dissolution Apparatus II in 900 ml of 0.1 N HCl at a temperature of 37±0.5° C. and paddles rotated at 50 rpm. As seen from the results of Table 2, the compositions according to invention exhibited immediate release profile of the active ingredient.

TABLE 2
Dissolution profile of compositions according to the invention

| Time | % Drug release | | |
|---|---|---|---|
| (minutes) | Example 1 | Example 2 | Example 3 |
| 5 | 69 | 46 | 28 |
| 10 | 90 | 88 | 78 |
| 15 | 96 | 97 | 95 |
| 20 | 99 | 100 | 98 |
| 30 | 100 | 102 | 99 |
| 45 | 101 | 103 | 99 |
| 60 | 101 | 103 | 100 |

The compositions according to invention were also tested for stability up to six months at temperature of 40° C. and a relative humidity of 75%. The results of the stability studies are provided in Tables 3 to 5.

TABLE 3
Stability data of composition according to Example 1

| Parameter | Initial | 40° C./75% RH | | |
|---|---|---|---|---|
| Months | 0 Month | 1 Month | 2 Month | 3 Month |
| Assay | 100.8 | 99.2 | 100.1 | 101.4 |

Dissolution Method: 0.1N HCl, 900 ml, USP II, 50 rpm

| Time (minutes) | % drug dissolved | | | |
|---|---|---|---|---|
| 5 | 69 | 68 | 57 | 62 |
| 10 | 90 | 89 | 90 | 86 |
| 15 | 96 | 97 | 98 | 94 |
| 20 | 99 | 100 | 100 | 97 |
| 30 | 100 | 101 | 101 | 99 |
| 45 | 101 | 102 | 102 | 99 |
| 60 | 101 | 103 | 102 | 99 |

Related Substances (% w/w) (By HPLC)

| | | | | |
|---|---|---|---|---|
| Substance A | 0.228 | 0.331 | 0.378 | 0.386 |
| Substance B | 0.04 | 0.04 | 0.024 | 0.05 |
| Substance C | 0.045 | 0.061 | 0.064 | 0.068 |
| Substance D | 0.007 | 0.009 | 0 | 0 |
| Highest | 0.049 | 0.041 | 0.018 | 0.017 |
| Unknown | | | | |
| Impurity | | | | |
| Total Unknown | 0.071 | 0.041 | 0.018 | 0.039 |
| Impurity | | | | |
| Total Related | 0.384 | 0.482 | 0.484 | 0.543 |
| Substances | | | | |

TABLE 4
Stability data of composition according to Example 2

| Parameter | Initial | 40° C./75% RH | | |
|---|---|---|---|---|
| Months | 0 Month | 1 Month | 2 Month | 3 Month |
| Assay | 103.9 | 101.2 | 102.3 | 101.2 |

Dissolution Method: 0.1N HCl, 900 ml, USP II, 50 rpm

| Time (minutes) | % drug dissolved | | | |
|---|---|---|---|---|
| 5 | 46 | 48 | 35 | 51 |
| 10 | 88 | 80 | 79 | 92 |
| 15 | 97 | 95 | 92 | 98 |
| 20 | 100 | 99 | 98 | 101 |
| 30 | 102 | 101 | 98 | 102 |
| 45 | 103 | 101 | 100 | 102 |
| 60 | 103 | 101 | 98 | 100 |

Related Substances (% w/w) (By HPLC)

| | | | | |
|---|---|---|---|---|
| Substance A | 0.126 | 0.242 | 0.336 | 0.401 |
| Substance B | 0.03 | 0.041 | 0.042 | 0.036 |
| Substance C | 0.039 | 0.047 | 0.054 | 0.061 |
| Substance D | 0 | 0.007 | 0.006 | 0.008 |
| Highest Unknown | 0.013 | 0.008 | 0.027 | 0.011 |
| Impurity | | | | |
| Total Unknown | 0.021 | 0.023 | 0.053 | 0.032 |
| Impurity | | | | |
| Total Related | 0.216 | 0.353 | 0.485 | 0.53 |
| Substances | | | | |

TABLE 5
Stability data of composition according to Example 3

| Parameter | Initial | 40° C./75% RH | | | |
|---|---|---|---|---|---|
| Months | 0 Month | 1 Month | 2 Month | 3 Month | 6 Month |
| Assay | 99.7 | 100.8 | 97.2 | 99.6 | 100.7 |

Dissolution Method: 0.1N HCl, 900 mL, USP II, 50 rpm

| Time (minutes) | % drug dissolved | | | | |
|---|---|---|---|---|---|
| 5 | 28 | 22 | 22 | 38 | 19 |
| 10 | 78 | 64 | 62 | 80 | 50 |
| 15 | 95 | 87 | 89 | 95 | 81 |
| 20 | 98 | 95 | 99 | 99 | 95 |
| 30 | 99 | 99 | 103 | 100 | 99 |
| 45 | 99 | 101 | 104 | 100 | 101 |
| 60 | 100 | 101 | 104 | 101 | 101 |

Related Substances (% w/w) (By HPLC)

| | | | | | |
|---|---|---|---|---|---|
| Substance A | 0.377 | 0.446 | 0.464 | 0.606 | 1.385 |
| Substance B | 0 | 0.025 | 0.023 | 0.011 | 0.022 |
| Substance C | 0.287 | 0.255 | 0.262 | 0.262 | 0.413 |
| Substance D | 0 | 0 | 0 | 0 | 0 |
| Highest | 0.082 | 0.015 | 0.029 | 0.024 | 0.014 |
| Unknown | | | | | |
| Impurity | | | | | |
| Total Unknown | 0.082 | 0.026 | 0.042 | 0.040 | 0.036 |
| Impurity | | | | | |
| Total Related | 0.746 | 0.752 | 0.791 | 0.919 | 1.856 |
| Substances | | | | | |

1. A stable pharmaceutical composition comprising a compound of Formula (I) or a stereoisomer or a pharmaceutically acceptable derivative thereof, and one or more pharmaceutically acceptable excipients.

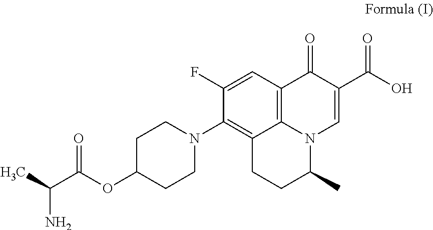 [SMILES: C[C@H](N)C(=O)OC1CCN(c2c(F)cc3c(=O)c(C(=O)O)cn4c3c2CC[C@@H]4C)CC1]

2. The pharmaceutical composition according to claim 1, wherein a compound of Formula (I) is present as L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate.

3. The pharmaceutical composition according to any one of claim 1 or 2, comprising less than about 2% w/w of total impurity following storage for six months at a temperature of 40° C. and a relative humidity of 75%.

4. The pharmaceutical composition according to any one of claim 1 or 2, comprising less than about 2% w/w of S-(−)-9-fluoro-8-(4-hydroxy-piperidin-1-yl)-5-methyl-6,7-dihydro-1-oxo-1H,5H-benzo[i,j] quinolizine-2-carboxylic acid following storage for six months at a temperature of 40° C. and a relative humidity of 75%.

5. The pharmaceutical composition according to any one of claim 1 or 2, comprising less than about 0.5% w/w of (S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%.

6. The pharmaceutical composition according to any one of claim 1 or 2, comprising less than about 0.5% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt following storage for six months at a temperature of 40° C. and a relative humidity of 75%.

7. The pharmaceutical composition according to any one of claim 1 or 2, comprising less than about 0.1% w/w of (S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j] quinolizine-2-carboxylic acid following storage for six months at a temperature of 40° C. and a relative humidity of 75%.

8. The pharmaceutical composition according to any one of claim 1 or 2, comprising the following:
(i) less than about 2% w/w of S-(−)-9-fluoro-8-(4-hydroxy-piperidin-1-yl)-5-methyl-6,7-dihydro-1-oxo-1H,5H-benzo[i,j] quinolizine-2-carboxylic acid;
(ii) less than about 0.5% w/w of (S)-(−)-8-(4-L-alaninyl oxypiperidin-1-yl)-5-methyl-6,7-dihydro-1-oxo-1H,5H-benzo[i,j] quinolizine-2-carboxylic acid, methane sulfonic acid salt;
(iii) less than about 0.5% w/w of (S)-(−)-9-fluoro-8-(4-D-alaninyl oxypiperidin-1-yl)-5-methyl-6,7-dihydro-1-oxo-1H,5H-benzo[i,j] quinolizine-2-carboxylic acid, methane sulfonic acid salt; and
(iv) less than about 0.1% w/w of (S)-(−)-9-fluoro-8-(4-(N-tert-butyloxy carbonyl-L-alaninyl)-oxypiperidin-1-yl)-5-methyl-6,7-dihydro-1-oxo-1H,5H-benzo[i,j]quinolizine-2-carboxylic acid;
following storage for six months at a temperature of 40° C. and a relative humidity of 75%.

9. The pharmaceutical composition according to any one of claim 1 or 2, wherein the compound of Formula (I) or a stereoisomer or a pharmaceutically acceptable derivative thereof is present in the composition in an amount of about 0.1 gram to about 10 gram.

10. The pharmaceutical composition according to any one of claims 1 to 9, wherein the composition is adapted for oral administration.

11. The pharmaceutical composition according to any one of claims 1 to 10, wherein the composition is in dosage form of a tablet, capsule, powder, granules, discs, caplets, pellets, granules in capsule, minitablets, minitablets in capsule or pellets in capsule.

12. The pharmaceutical composition according to any one of claims 1 to 11, wherein the composition is in form of a tablet.

13. The pharmaceutical composition according to any one of claims 10 to 12, wherein the composition exhibits a dissolution profile such that about 50% or more of a compound of Formula (I) or a stereoisomer or a pharmaceutically acceptable derivative thereof is released within 15 minutes, when measured using a USP Dissolution Apparatus II in 900 ml of 0.1 N HCl at a temperature of 37±0.5° C. and 50 rpm.

14. The pharmaceutical composition according to any one of claims 10 to 12, wherein the composition exhibits a dissolution profile such that about 75% or more of a compound of Formula (I) or a stereoisomer or a pharmaceutically acceptable derivative thereof is released within 20 minutes, when measured using a USP Dissolution Apparatus II in 900 ml of 0.1 N HCl at a temperature of 37±0.5° C. and 50 rpm.

15. The pharmaceutical composition according to any one of claims 1 to 14, wherein compound of Formula (I) is having d₉₀ particle size of equal to or less than 150 μm.

16. A process for preparing the composition according to any one of claims 12 to 15, in form of tablets; the process comprising:
(a) mixing a compound of Formula (I) or a stereoisomer or a pharmaceutically acceptable derivative thereof with one or more diluents and one or more disintegrants;
(b) wet granulating the mixture of step (a) in presence of a binder solution;
(c) drying and sieving the granulated mixture obtained in step (b);
(d) optionally blending the granulated mixture obtained in step (c) with one or more of a diluent, binder, disintegrant, glidant and lubricant;
(e) compressing the mixture obtained in step (c) or step (d) into tablets; and
(f) optionally film coating the tablets.

17. A process for preparing the composition according to any one of claims 12 to 15, in form of tablets; the process comprising:
(a) mixing L-alanine, 1-[(5S)-2-carboxy-9-fluoro-6,7-dihydro-5-methyl-1-oxo-1H,5H-benzo[i,j]quinolizin-8-yl]-4-piperidinyl ester, methanesulfonate with one or more diluents and one or more disintegrants;
(b) wet granulating the mixture of step (a) in presence of a binder solution;
(c) drying and sieving the granulated mixture obtained in step (b);
(d) optionally blending the granulated mixture obtained in step (c) with one or more of a diluent, binder, disintegrant, glidant and lubricant;
(e) compressing the mixture obtained in step (c) or step (d) into tablets; and
(f) optionally film coating the tablets.

18. The pharmaceutical composition according to any one of the claims 1 to 15 for use in treatment or prevention of bacterial infections.

19. A method for treating bacterial infections in a subject comprising administering to the subject a composition according to any one of claims 1 to 15.
